(19) United States
(12) Patent Application Publication
(10) Pub. No.: US 2005/0163748 A1
(43) Pub. Date: Jul. 28, 2005

(54) Anti-adhesion balloons

(76) Inventor: Bret A. Ferree, Cincinnati, OH (US)

Correspondence Address: John G. Posa;Gifford, Krass, Groh, Sprinkle,, Anderson & Citkowski, P.C., PO Box 7021, Troy, MI, 48007-7021, US

(21) Appl. No.: 11/041,647

(22) Filed: Jan. 24, 2005

Related U.S. Application Data
(60) Provisional application No. 60/538,185, filed on Jan. 22, 2004.

(51) Int. Cl. A61K 38/19; A61K 38/18; A61M 31/00

(52) U.S. Cl. 424/85.1; 514/2; 604/500

(57) ABSTRACT

An anti-adhesion device in the form of a fillable balloon having a wall structure permits suturing or other attachment of the balloon to surrounding tissues. The balloon may be filled with a gas, liquid, or gel, and may be constructed of a resorbable material. The balloon may contain one or more growth factors and one or more antibodies to the growth factors may also be provided. Cytokines may be incorporated into the wall structure. The associated surgical procedure includes suturing or otherwise attaching the balloon between healing tissues and surrounding tissues. The healing tissues may include the vagina and the surrounding tissues include the small intestines, the great vessels and the spine following anterior spinal surgery, or any other appropriate location(s). The balloon may be placed immediately after an open surgical procedure or, alternatively, through smaller incisions as part of a minimally invasive procedure. Thus, the balloon may be filled prior to introduction or following placement. In the preferred embodiment the balloon ruptures and resorbs after the surgically treated tissues are partly or completely healed.

### REFERENCE TO RELATED APPLICATION

This application claims priority from U.S. provisional Patent Application Ser. No. 60/538,185, filed Jan. 22, 2004, the entire content of which is incorporated herein by reference.

### FIELD OF THE INVENTION

This invention relates generally to surgical instruments and procedures and, in particular, to inflatable structures that reduce adhesions.

### BACKGROUND OF THE INVENTION

Adhesions often form between the small intestines and surrounding tissues. Adhesions are particularly common following hysterectomies. Gravity pulls the small intestines into the pelvis. The intestines can become attached to the healing tissues in the pelvis following removal of the uterus. Adhesions can cause bowel obstructions. Bowel obstructions can lead to additional surgeries, pain, and even death.

### SUMMARY OF THE INVENTION

This invention improves upon the prior art by providing an anti-adhesion device in the form of a fillable balloon having a wall structure. One or more portions are provided on the balloon to permit suturing or other attachment of the balloon to surrounding tissues.

The balloon may be filled with a gas, liquid, or gel, and may be constructed of a resorbable material. The balloon may contain one or more growth factors, including TGF-Beta, Platelet Derived Growth Factor, or Vascular Endothelial Growth Factor. One or more antibodies to the growth factors may also be provided. Cytokines may be incorporated into the wall structure.

The associated surgical procedure includes suturing or otherwise attaching the balloon between healing tissues and surrounding tissues. The healing tissues may include the vagina and the surrounding tissues include the small intestines, the great vessels and the spine following anterior spinal surgery, or any other appropriate location(s).

The balloon may be placed immediately after an open surgical procedure or, alternatively, through smaller incisions as part of a minimally invasive procedure. Thus, the balloon may be filled prior to introduction or following placement. In the preferred embodiment the balloon ruptures and resorbs after the surgically treated tissues are partly or completely healed.

### BRIEF DESCRIPTION OF THE DRAWINGS

FIG. 1 is an anterior view of a balloon according to the present invention;

FIG. 2 is a cross section of the novel balloon; and

FIG. 3 is sagittal cross section of the pelvis of a female.

### DETAILED DESCRIPTION OF THE INVENTION

According to this invention, a balloon according to the invention is placed between healing tissues and surrounding tissues. For example, the balloon can be placed between the tissues that are sutured following hysterectomy and the small intestines. The balloon prevents the small intestines from entering the pelvis. The balloon is made of a resorbable material in the preferred embodiment of the invention. The balloon ruptures and resorbs after the surgically treated tissues are partly or completely healed. The balloon may be placed immediately after an open surgical procedure or, alternatively, through smaller incisions as part of a minimally invasive procedure. Thus, the balloon may be filled prior to introduction or following placement.

The balloon can contain growth factors such as TGF-Beta, Platelet Derived Growth Factor, Vascular Endothelial Growth Factor, etc. The cytokines can also be incorporated into the walls of the balloon. Antibodies to the growth factors can also be incorporated into the balloon. The novel balloon can be used in other areas of the body. For example, the balloon can be placed between the great vessels and the spine following anterior spinal surgery.

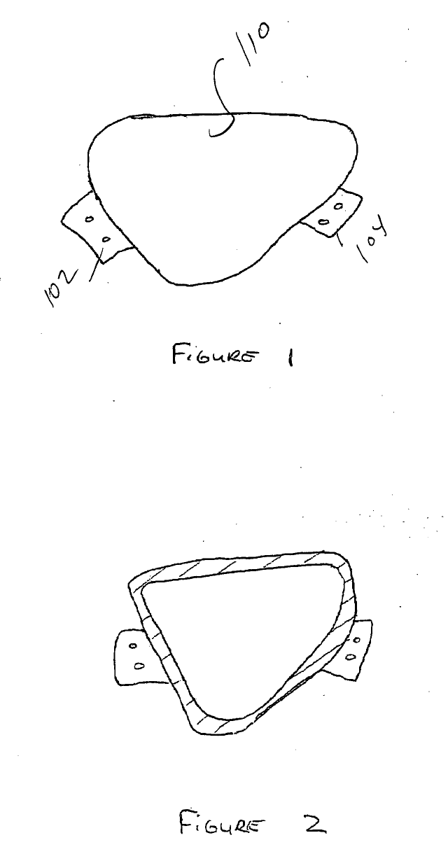

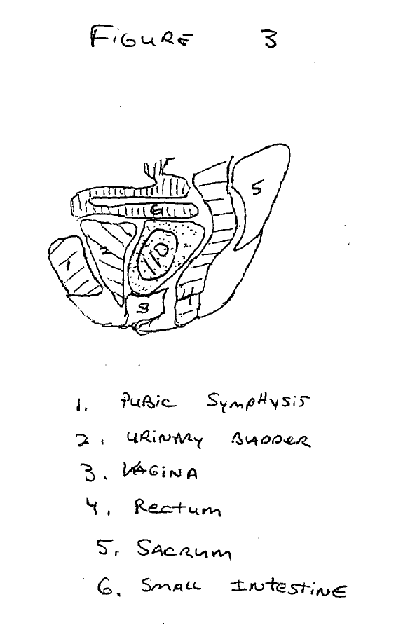

FIG. 1 is an anterior view of a balloon according to the invention. Flaps 102, 104 extending from the sides of the balloon 110 can be sutured to the surrounding tissues. FIG. 2 is a cross section of the novel balloon. The balloon can be filled with air or other gasses, liquids, or gel(s).

FIG. 3 is sagittal cross section of the pelvis of a female. The balloon 110 can be seen between the vagina and the small intestines. The balloon could be placed in location shown in FIG. 3 following hysterectomy. The balloon prevents contact between the sutured tissues above the vagina and the small intestines until the sutured tissues are at least partially healed.

1. An anti-adhesion device, comprising:
a fillable balloon having a wall structure; and
one or more portions that permit suturing or other attachment of the balloon to surrounding tissues.

2. The an anti-adhesion device of claim 1, wherein the balloon is filled with a gas, liquid, or gel.

3. The an anti-adhesion device of claim 1, wherein the balloon is constructed of a resorbable material.

4. The an anti-adhesion device of claim 1, wherein the balloon contains one or more growth factors.

5. The an anti-adhesion device of claim 1, wherein the growth factor is TGF-Beta.

6. The an anti-adhesion device of claim 1, wherein the growth factor is Platelet Derived Growth Factor.

7. The an anti-adhesion device of claim 1, wherein the growth factor is Vascular Endothelial Growth Factor.

8. The an anti-adhesion device of claim 1, further including one or more antibodies to the growth factors.

9. The an anti-adhesion device of claim 1, further including cytokines incorporated into the wall structure.

10. A method of reducing or preventing adhesions, comprising the steps of:
providing the balloon of claim 1; and
suturing or otherwise attaching the balloon between healing tissues and surrounding tissues.

11. The method of claim 10, wherein the healing tissues include the vagina and the surrounding tissues include the small intestines.

12. The method of claim 10, wherein the balloon is placed between the great vessels and the spine following anterior spinal surgery.

13. The method of claim 10, wherein the balloon ruptures and resorbs after the surgically treated tissues are partly or completely healed.
